(19) United States
(12) Patent Application Publication
(10) Pub. No.: US 2007/0077217 A1
(43) Pub. Date: Apr. 5, 2007

(54) Cosmetic compositions having increased color intensity and method for producing same

(76) Inventors: Ginger King, Valley Cottage, NY (US); Hung-Ta Lin, Fort Lee, NJ (US)

Correspondence Address: AVON PRODUCTS, INC., AVON PLACE, SUFFERN, NY, 10901, US

(21) Appl. No.: 11/242,480

(22) Filed: Oct. 3, 2005

(51) Int. Cl. A61K 8/37

(52) U.S. Cl. 424/63

(57) ABSTRACT

Color intensity of a pigment containing cosmetic composition is increased by incorporating in the composition a color intensifying amount of:
(i) a C₁₂-C₂₄ fatty alcohol, preferably a C₁₄-C₂₀ fatty alcohol, the fatty alcohol preferably being a branched chain fatty alcohol having a C₆-C₁₀ alkyl branch and
(ii) an unsaturated dimerized or trimerized C₁₄-C₂₄ fatty acid, preferably an unsaturated dimerized or trimerized C₁₆-C₂₀ fatty acid that has 2 to 4, preferably 2, unsaturated bonds. Diisostearyl dilinoleate is preferred. Triisostearyl trilinoleate is most preferred. When then composition is an oil-in-water emulsion, the triisostearyl trilinoleate or diisostearyl dilinoleate is added to the emulsion after it is prepared in order to maximize color intensification.

### FIELD OF THE INVENTION

This invention relates to cosmetic compositions containing a pigment colorant. In particular this invention relates to a method of increasing the color intensity of a pigment colorant containing composition, and compositions useful in the practice of the method.

### BACKGROUND OF THE INVENTION

Cosmetic compositions containing pigments are well known in the art. Such compositions include, for example, mascara, makeup, rouge, eye shadow, eyeliner, hair dye, facial gloss, hair gloss, skin creams and the like.

As compared to other ingredients typically employed in such cosmetic compositions, pigments are expensive. Additionally, some users of such compositions may exhibit skin sensitivity to pigments.

To reduce composition cost and decrease the possibility of skin sensitivity to pigment containing compositions, it would be desirable to reduce the amount of pigment contained in such compositions without adversely affecting the color intensity of such compositions. Alternatively, it would be desirable to increase the intensity of the color obtained for a predetermined amount of the pigment.

In the prior art, silicones have been employed in order to boost shine. However, silicones are disadvantageous as they are expensive. More importantly, they may not be compatible with cosmetic products that are water based.

### SUMMARY OF THE INVENTION

The present invention provides a method for increasing color intensity of a pigment-containing cosmetic composition comprising incorporating in such composition an esterification reaction product of:
(i) a C₁₂-C₂₄ fatty alcohol, preferably a C₁₄-C₂₀ fatty alcohol, the fatty alcohol preferably being a branched chain fatty alcohol having a C₆-C₁₀ alkyl branch and
(ii) an unsaturated dimerized or trimerized C₁₄-C₂₄ fatty acid, preferably an unsaturated dimerized or trimerized C₁₆-C₂₀ fatty acid that has 2 to 4, preferably 2, unsaturated bonds,
   in an amount effective to increase the color intensity of the composition, as compared to a like composition not containing an esterification reaction product of the present invention.

Preferred esterification reaction products employable in the present invention have the following structural Formula I

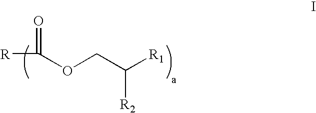

wherein R is the aliphatic hydrocarbon residue of a dimerized or trimerized C₁₆ to C₁₈ fatty acid;
R₁ and R₂ are independently a linear alkyl of 6 to 9 carbons;
a is 2 when R is a dimer acid radical, and
a is 3 when R is a trimer acid radical.
   The preferred compound of Formula I useful to increase pigment color intensity is triisostearyl trilinoleate.

The present invention also provides a cosmetic composition comprising a pigment, a cosmetically acceptable vehicle therefore and an amount of an esterification reaction product of the present invention:
(i) a C₁₂-C₂₄ fatty alcohol, preferably a C₁₄-C₁₈ fatty alcohol, the fatty alcohol preferably being a branched chain fatty alcohol having a C₆-C₁₀ alkyl branch and
(ii) an unsaturated dimerized or trimerized C₁₄-C₂₄ fatty acid, preferably an unsaturated dimerized or trimerized C₁₆-C₁₈ fatty acid that has 2-4, preferably 2, unsaturated bonds,
   preferably a compound of Formula I, particularly triisostearyl trilinoleate, effective to increase color intensity of the pigment in the composition, as compared to a like composition not containing the an esterification reaction product of the present invention.

Still further, the present invention provides an improvement to a cosmetic composition containing a pigment in a cosmetically acceptable aqueous based vehicle, the pigment in said vehicle exhibiting a first color intensity, wherein the improvement comprises incorporating in the composition an amount of an esterification reaction product:
(i) a C₁₂-C₂₄ fatty alcohol, preferably a C₁₄-C₂₀ fatty alcohol, the fatty alcohol preferably being a branched chain fatty alcohol having a C₆-C₁₀ alkyl branch and
(ii) an unsaturated dimerized or trimerized C₁₄-C₂₄ fatty acid, preferably an unsaturated dimerized or trimerized C₁₆-C₂₀ fatty acid that has 2 to 4, preferably 2, unsaturated bonds,
   preferably a compound of Formula I, particularly triisostearyl trilinoleate, so that the resulting composition exhibits a second color intensity that is greater than the first color intensity.

Additionally, the present invention provides a process for increasing color intensity of a pigment dispersion in an oil-in-water emulsion based cosmetic composition comprising producing an oil-in-water emulsion containing the pigment, and thereafter incorporating a an esterification reaction product:
(i) a C₁₂-C₂₄ fatty alcohol, preferably a C₁₄-C₂₀ fatty alcohol, the fatty alcohol preferably being a branched chain fatty alcohol having a C₆-C₁₀ alkyl branch and (ii) an unsaturated dimerized or trimerized C₁₄-C₂₄ fatty acid, preferably an unsaturated dimerized or trimerized C₁₆-C₂₀ fatty acid that has 2 to 4, preferably 2, unsaturated bonds, preferably a compound of Formula I, particularly triisostearyl trilinoleate, in the aqueous phase of the emulsion, in an amount effective to increase color intensity of the pigment as compared to a like composition not containing the esterification reaction product.

The present invention further provides a process for increasing shine of a pigment dispersion in an oil-in-water emulsion containing the pigment by incorporating therein an esterification reaction product of the present invention, especially a compound of Formula I, particularly triisostearyl trilinoleate, in the aqueous phase of the emulsion, in an amount effective to increase shine as compared to a like composition not containing said reaction product, the compound of Formula I, or particularly triisostearyl trilinoleate.

Moreover, the present invention provides a cosmetic composition comprising a pigment, a cosmetically acceptable oil-in-water emulsion based vehicle therefore and an amount of an esterification reaction product of the present invention, especially a compound of Formula I, particularly triisostearyl trilinoleate, effective to increase shine as compared to a like composition not containing said reaction product, the compound of Formula I, or particularly triisostearyl trilinoleate.

Still further, the present invention provides an improvement to a cosmetic composition containing a pigment in a cosmetically acceptable oil-in-water emulsion based vehicle therefore, wherein the improvement comprises incorporating in the aqueous phase of the oil-in-water emulsion based vehicle, an amount of an esterification reaction product of the present invention, especially a compound of Formula I, particularly triisostearyl trilinoleate, so that the composition has a higher gloss than a like composition not containing said reaction product, the compound of Formula I, or particularly triisostearyl trilinoleate.

Prior to the present invention it was not appreciated that an esterification reaction product of the present invention, particularly a compound of Formula I, could be incorporated in a cosmetic composition containing one or more pigments in order to boost the color intensity of the pigment(s) and/or impart shine to the composition. Nor did the prior art appreciate that the esterification reaction product, such as the compound of Formula I, should be added to such compositions at a particular point in the process of manufacturing such composition in order to achieve the highest boost in pigment color intensity.

### DETAILED DESCRIPTION OF THE INVENTION

The present invention comprises incorporating in a cosmetic composition containing a pigment, a color intensity boosting amount of an esterification reaction product of:
(i) a C₁₂-C₂₄ fatty alcohol, preferably a C₁₄-C₂₀ fatty alcohol, the fatty alcohol preferably being a branched chain fatty alcohol having a C₆-C₁₀ alkyl branch and
(ii) an unsaturated dimerized or trimerized C₁₄-C₂₄ fatty acid, preferably an unsaturated dimerized or trimerized C₁₆-C₂₀ fatty acid that has 2 to 4, preferably 2, unsaturated bonds.
   Preferably esterification reaction products has the following structural Formula I:

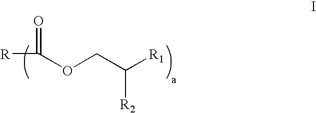

wherein R is the aliphatic hydrocarbon residue of a dimerized or trimerized C₁₆ to C₁₈ fatty acid;
R₁ and R₂ are independently a linear alkyl of 6 to 9 carbons;
a is 2 when R is a dimer acid radical, and
a is 3 when R is a trimer acid radical.
   The compound of Formula I wherein a is 2, R₁ is n-C₉—H₁₉, R₂ is n-C₇H₁₅, and R is a dilinoleayl group is diisostearyl dilinoleate (hereinafter referred to as “DISD”).
   The compound of Formula I wherein a is 3, R₁ is n-C₉—H₁₉, R₂ is n-C₇H₁₅, and R is a trilinoleayl group is triisostearyl trilinoleate.

TIST is available from Scher Chemicals as Schercemol TIST. Its INCI name is triisostearyl trilinoleate. It is the triester of isostearyl alcohol and trilinoleic acid and has the structural formula Ia:

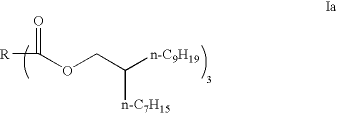

wherein R is a trilinoleayl group. TIST is the most preferred esterication reaction product in accordance with this invention.

TIST has been employed in the prior art as a dispersant for coloring agents, a wetting agent and as a binder for pigments and pearlescent agents. It has been recommended as a replacement for lanolin oil.

DISD is available from Scher Chemicals a Schercemol DISD. It is the diester of isostearyl alcohol and dilinoleic acid and has the structural formula Ib:

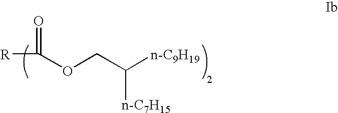

wherein R is a dilinoleayl group.

It is indeed surprising and unexpected that an esterification reaction product of the present invention, particularly a compound of Formula I such as TIST, can be employed to dramatically boost color intensity and/or impart shine. Advantageously, a compound of Formula I, particularly TIST, can be employed to boost color intensity and/or impart shine in water-based pigment containing cosmetic products, particularly pigment containing oil-in-water emulsion based cosmetic products, aqueous gels, aqueous lotions, etc.

It should be noted that color intensity is measured with a Minolta CM-2600d Spectrophotomer and is defined by the L*a*b* values so determined.

The following method was employed to determine black intensity of a mascara composition.
1. Using draw down applicators (having a thickness of 15 mm) samples were drawn down as unit films on glass slides. They were permitted to dry for 30 minutes.
2. A Minolta CM-2600d Spectrophotomer (a broad range of color measuring system) was employed to collect black intensity data. Six (6) points were read on each slide. Ten (10) different samples of the mascara were drawn down on 10 different slides giving a total of 60 data points.

L*a*b* color space is one of the most widely used and useful of the color models. L*a*b* is device independent and represents every color through three (3) components. The L value represents luminance (lightness) and ranges in uniform steps from 0 for black to 100 for white. The a and the b values are represented as +a/−a for red/green and +b/−b for blue/yellow. L* is employed to determine black intensity.

The present inventors have found that although color is intensified when the compound of the Formula Ib wherein R is a dimer acid radical (“DISD”) is employed, it is intensified to a lesser extent than when the compound of Formula Ia, wherein R is trimer acid radical (“TIST”), is employed.

To boost color intensity and/or impart shine, triisostearyl trilinoleate is generally employed in a concentration of from 0.1 to 10% by weight, based on the total weight of the composition. In one embodiment it is employed in a concentration of from 0.25 to 5% by weight, based on the total weight of the composition. With the pigments tested at (see the Examples which follow) 0.25% by weight, based on the total weight of the composition, was found to be a desirable concentration. It should be noted that with many compositions a concentration greater than 2% by weight, based on the total weight of the composition, may increase viscosity as well as increase the cost of the final composition.

Initially the present inventors postulated that the color intensifying action of TIST could well be attributable to its high refractive index. However, other materials having high refractive index were tested and were found not significantly enhance color intensity. Such tested agents included mica, phenyltrimethicone and dimethylpolysiloxane.

Thus, some other mechanism, or combination of mechanisms (including high refractive index) is most likely involved.

Pigments employable in the present invention include, but are not limited to, black iron oxide, ultramarines, red iron oxide, yellow iron oxide, titanium dioxide, zinc oxide cobalt aluminum oxide, chromium oxide green, chromium hydroxide, and D&C Black 2 (carbon black).

Pigments, as used herein, also include lake pigments such as, for example, D&C Red 7 Ca Lake, FD&C Yellow 5 Al Lake, FD&C Blue Al Lake, D&C Red 6 Ba Lake, D&C Red 33 Al Lake, FD&C Red 3 Al Lake, D&C Red 27 Al Lake, D&C Red 21 Al Lake, D&C C Red 7 Na Lake, FD&C Yellow 6 Al Lake, D&C Orange Zr Lake, D&C Red 19 Al Lake and D&C Red 21 Al Lake.

The present invention will now be described with reference to the Examples that follow in which, unless otherwise indicated, all percentages are percent by weight and are based on the total weight of the composition.

### EXAMPLE 1

### Black Mascara

A black mascara composition having the following formulation was prepared.

| Ingredient | % |
|---|---|
| Deionized water | q.s. 100 |
| Triethanolamine | 1.5 |
| EDTA | 0.1 |
| Hydroxyethylcellulose | 1.0 |
| Polyvinyl alcohol | 5.5 |
| Methylparaben | 0.38 |
| Propylparaben | 0.2 |
| Oleth-3 phosphate | 0.5 |
| Isoceteth-20 | 0.5 |
| C₁₈₋₃₆ acid triglyceride | 1.0 |
| Palmitic acid | 4.0 |
| Stearic acid | 2.0 |
| Black iron oxide pigment | 45.0 |
| dispersion | |
| Acrylate copolymer | 5.0 |
| Phenoxyethanol | 0.4 |
| Isododecane | 1.0 |
| Isopentane | 2.0 |
| TIST | 0.25 |

The composition was prepared as follows:
1. The formulation amounts of deionized water, triethanolamine, EDTA, hydroxyethylcellulose, polyvinyl alcohol and methyl paraben were mixed and heated to 80° C.
2. The formulation amounts of oleth-3 phosphate, isoceteth-20, propyl paraben, C₁₈₋₃₆ acid triglyceride, palmitic acid and stearic acid were mixed and heated to 80° C.
3. The mixture produced in Step 2 was added to the aqueous mixture produced in Step 1 with high speed mixing and the resultant emulsion was cooled to 45° C.
4. The remaining formulation ingredients were then mixed into the batch, with TIST being added last.

A second black mascara formulation was prepared in the same manner as a control. It was identical to the first formulation except that it did not contain TIST.

The L* value was determined for each of the compositions. The black mascara composition employed as a control exhibited an L* (D65) value of 23.28. The composition exhibited a color intensity of black. The mascara composition to which 0.25% TIST was added was determined to have an L*(D65) value of 20.63 It exhibited a color intensity of dark black.

### EXAMPLE 2

### Navy Mascara

A navy mascara composition having the following formulation was prepared in accordance with process described in Example 1.

| Name | % |
|---|---|
| Deionized water | q.s. 100 |
| Triethanolamine | 1.5 |
| EDTA | 0.1 |
| Hydroxyethylcellulose | 1.0 |
| Polyvinyl alcohol | 5.5 |
| Methylparaben | 0.38 |
| Propylparaben | 0.2 |
| Oleth-3 phosphate | 0.5 |
| Isoceteth-20 | 0.5 |
| C18-36 acid triglyceride | 1.0 |
| Palmitic acid | 4.0 |
| Stearic acid | 2.0 |
| Ultramarine blue pigment | 45.0 |
| dispersion | |
| Acrylate copolymer | 5.0 |
| Phenoxyethanol | 0.4 |
| Isododecane | 1.0 |
| Isopentane | 2.0 |
| TIST | 0.25 |

A second navy mascara formulation was prepared in the same manner as a control. It was identical to the first formulation except that it did not contain TIST.

The L* value was determined for each of the compositions. The mascara composition employed as a control exhibited a b* (D65) value of −9.55. The composition had a color intensity of navy blue. The mascara composition containing 0.25% TIST was determined to have a b*(D65) value of −10.89. It exhibited a color intensity of deep navy blue.

### EXAMPLE 3

### Black Mascara

A black mascara composition containing 2% TIST and having the following formulation was prepared in accordance with the process described in Example 1:

| Ingredient | % |
|---|---|
| Deionized water | q.s. 100 |
| Triethanolamine | 1.5 |
| EDTA | 0.1 |
| Hydroxyethylcellulose | 1.0 |
| Polyvinyl alcohol | 5.5 |
| Methylparaben | 0.38 |
| Propylparaben | 0.2 |
| Oleth-3 phosphate | 0.5 |
| Isoceteth-20 | 0.5 |
| C₁₈₋₃₆ acid triglyceride | 1.0 |
| Palmitic acid | 4.0 |
| Stearic acid | 2.0 |
| Black iron oxide pigment | 45.0 |
| dispersion | |
| Acrylate copolymer | 5.0 |
| Phenoxyethanol | 0.4 |
| Isododecane | 1.0 |
| Isopentane | 2.0 |
| TIST | 2.0 |

A second black mascara formulation was prepared in the same manner, as a control. It was identical to the first formulation except that it did not contain TIST.

The L* value determined for each of the compositions. The second black mascara composition employed as a control was black in intensity and had an L*(D65) value of 28.97. The first black mascara composition containing 2% TIST was dark black in intensity and had an L*(D65) value of 25.27.

### EXAMPLE 4

### Blue Mascara

A blue mascara composition containing 2% TIST and was prepared in accordance with the process described in Example 1:

The formulation was identical to that employed in Example 1 except it contained 2% TIST, and the pigment employed was Ultramarine Blue.

A second blue mascara formulation was prepared in the same manner, as a control. It was identical to the first formulation expect that it did not contain TIST.

The b* value was determined for each of the compositions.

The second blue mascara composition, employed as a control, was blue in intensity and had a b*(D65) value of −33.86.

The first blue mascara composition, containing 2% TIST, was deep blue in intensity and had a b*(D65) value of −42.06.

### EXAMPLE 5

### Brown Mascara

A brown mascara composition identical to that employed in Example 1, except that it contained 0.5% TIST, 33% black iron oxide dispersion, 6% red iron oxide dispersion and 6% yellow iron oxide dispersion instead of the 45% black iron oxide pigment present in Example 1, was prepared in accordance with the process described in Example 1.

A second brown mascara formulation was prepared in the same manner, as a control.

It was identical to the first formulation expect that it did not contain TIST and the water was increased 0.5% to compensate for the TIST.

The a*(D65) and b*(D65) values were determined for each of the compositions.

The second brown mascara composition, employed as a control, had an a*(D65) value of 2.79 and b*(D65) value of 2.55.

The first brown mascara composition, containing 0.5% TIST, had an a*(D65) value of 2.66 and ab*(D65) value of 2.35.

In addition to the surprising fact that TIST boosted color intensity of the pigment, the present inventors surprisingly found that TIST is most effective in increasing color intensity when it is added at or toward the end of the process of manufacturing the pigment containing composition. Most desirably, it is post added, in other words, at the end of the process for producing the composition.

What is important is that the equipment employed in the manufacturing process and the time of TIST addition in the manufacturing process be such as to ensure that the TIST is not emulsified as a component of the dispersed discontinuous oil phase. In order to obtain the maximum color intensifying effect, TIST, a water dispersible material, must be in the aqueous continuous phase.

The present inventors found that when TIST is incorporated in the internal phase of an oil-in-water emulsion based pigment containing cosmetic composition it does not intensify the color of the composition. It must be added to the external aqueous phase. This may well explain why the use of TIST for intensifying color of a pigment has not heretofore been appreciated by the prior art. As noted earlier, TIST has been used in the prior art as an emollient and as such has been employed as part of the oil phase in an oil-in-water emulsion. The present inventors have determined that when TIST is in the internal phase of an oil-in-water emulsion, it does not act as a color intensifier.

The following Example 6 illustrates the importance of the point of addition of the compound of Formula I in the manufacturing process.

### EXAMPLE 6

A mascara composition identical to the of Example 1 except that it contained 0.5% TIST was prepared in accordance with the process described in Example 1. In such process the TIST was added to the emulsion produced by mixing the oil phase and water phase. Thus it was the last added ingredient.

A like composition control was prepared in the same manner except that the TIST was added to the oil phase prior to mixing of the oil and water phases to produce the emulsion.

The L*(D65) value was determined for each of the compositions.

The composition in which the TIST was added to the oil phase prior to emulsification with the water phase had an L*(D65) value of 24.65.

In contrast thereto, the composition in which the TIST was added to the prepared emulsion had an L*(D65) value of 21.19. This is indeed surprising and unexpected.

### EXAMPLE 7

To determine a practical range for the use of TIST to intensify the color of a black pigment, black mascara compositions A-G were prepared and the L*(D65) value of each composition was determined. Compositions A-G were identical to the composition employed in Example 1 except that compositions A, B, C, D, E, F and G, respectively contained 0.25%, 0.5%, 1%, 2%, 5.5%, 10% and 0% TIST. The water content of the formulations was adjusted to compensate for the increase in TIST content.

Composition G, which contained no TIST, served as a control.

The L*(D65) values of compositions A-G were as follows:

| Composition | % TIST | L*(D65) |
|---|---|---|
| A | 0.25 | 20.63 |
| B | 0.5 | 21.30 |
| C | 1 | 21.75 |
| D | 2 | 21.64 |
| E | 5.5 | 21.61 |
| F | 10 | 22.23 |
| G | — | 23.28 |

### EXAMPLE 8

To determine a practical range for the use of DISD to intensity the color of a black pigment, black mascara compositions H-K were prepared and the L*(D65) value of each composition was determined. Compositions H-K were identical to the composition employed in Example 1 except that compositions H-K employed DISD instead of TIST. Additionally, compositions H, I, J, and K, respectively contained 0.25%, 0.5%, 2% and 0% DISD. The water content of the compositions was adjusted to compensate for the increase in DISD content.

Composition K, which contained no DISD, served as a control.

The L*(D65) values of compositions H-K were as follows:

| Composition | % DISD | L*(D65) |
|---|---|---|
| H | 0.25% | 21.86 |
| I | 0.5% | 22.06 |
| J | 2.0% | 22.65 |
| K | — | 23.28 |

### EXAMPLE 9

To determine a practical range for use of TIST to intensity the color of a blue pigment, blue mascara compositions L-O were prepared and the b*(D65) value for each composition was determined. Compositions L-O were identical to the composition employed in Example 4 except that compositions L-O respectively contained 0.25%, 0.5%, 1% and 0% TIST. The water content of the compositions was adjusted to compensate for the increase in TIST content.

Composition 0, which contained no TIST, served as a control.

The L*(D65) values of compositions L-O were as follows:

| Composition | % TIST | b*(D65) |
|---|---|---|
| L | 0.25% | −10.89 |
| M | 0.5% | −10.42 |
| N | 1.0% | −10.53 |
| O | — | −9.55 |

What is claimed is:

1. A method for increasing color intensity of a pigment containing an aqueous based cosmetic composition comprising incorporating in such composition an amount of an esterification reaction product of
(i) a C₁₂-C₂₄ fatty alcohol and
(ii) an unsaturated dimerized or trimerized C₁₄-C₂₄ fatty acid,
effective to increase color intensity of the composition as compared to a like composition not containing the esterification reaction product.

2. In a cosmetic composition containing a pigment and a cosmetically acceptable aqueous based vehicle therefore, the composition having a first color intensity, the improvement comprising incorporating in said composition an amount of an esterification reaction product of
(i) a C₁₂-C₂₄ fatty alcohol and
(ii) an unsaturated dimerized or trimerized C₁₄-C₂₄ fatty acid,
such that the resultant composition exhibits a second color intensity greater than the first color intensity.

3. A cosmetic composition comprising a pigment, an esterification reaction product of
(i) a C₁₂-C₂₄ fatty alcohol and
(ii) an unsaturated dimerized or trimerized C₁₄-C₂₄ fatty acid,
and a cosmetically aqueous acceptable vehicle therefor, the esterification reaction product being present in the composition in an amount such that the composition has a color intensity greater than the color intensity exhibited by a like composition not containing the esterification reaction product.

4. A process for increasing color intensity of a pigment dispersion in an oil-in-water emulsion based cosmetic composition comprising producing an oil-in-water emulsion containing said pigment and then incorporating an esterification reaction product of
(i) a C₁₂-C₂₄ fatty alcohol and
(ii) an unsaturated dimerized or trimerized C₁₄-C₂₄ fatty acid,
in the aqueous phase of the emulsion in an amount effective to increase color intensity of the pigment as compared to a like composition not containing the esterification reaction product.

5. The method as claimed in claim 1, wherein the pigment is selected from the group consisting of black iron oxide, ultramarines, red iron oxide, yellow iron oxide, titanium dioxide, zinc oxide cobalt aluminum oxide, chromium oxide green, chromium hydroxide, D&C Black #2, D&C Red 7 Ca Lake, FD&C Yellow 5 Al Lake, FD&C Blue Al Lake, D&C Red 6 Ba Lake, D&C Red 33 Al Lake, FD&C Red 3 Al Lake, D&C Red 27 Al Lake, D&C Red 21 Al Lake, D&C C Red 7 Na Lake, FD&C Yellow 6 Al Lake, D&C Orange Zr Lake, D&C Red 19 Al Lake and D&C Red 21 Al Lake and mixtures thereof.

6. The composition as claimed in claim 2, wherein the pigment is selected from the group consisting of black iron oxide, ultramarines, red iron oxide, yellow iron oxide, titanium dioxide, zinc oxide cobalt aluminum oxide, chromium oxide green, chromium hydroxide, D&C Black #2, D&C Red 7 Ca Lake, FD&C Yellow 5 Al Lake, FD&C Blue Al Lake, D&C Red 6 Ba Lake, D&C Red 33 Al Lake, FD&C Red 3 Al Lake, D&C Red 27 Al Lake, D&C Red 21 Al Lake, D&C C Red 7 Na Lake, FD&C Yellow 6 Al Lake, D&C Orange Zr Lake, D&C Red 19 Al Lake and D&C Red 21 Al Lake and mixtures thereof.

7. The composition as claimed in claim 3, wherein the pigment is selected from the group consisting of black iron oxide, ultramarines, red iron oxide, yellow iron oxide, titanium dioxide, zinc oxide cobalt aluminum oxide, chromium oxide green, chromium hydroxide, D&C Black #2, D&C Red 7 Ca Lake, FD&C Yellow 5 Al Lake, FD&C Blue Al Lake, D&C Red 6 Ba Lake, D&C Red 33 Al Lake, FD&C Red 3 Al Lake, D&C Red 27 Al Lake, D&C Red 21 Al Lake, D&C C Red 7 Na Lake, FD&C Yellow 6 Al Lake, D&C Orange Zr Lake, D&C Red 19 Al Lake and D&C Red 21 Al Lake and mixtures thereof.

8. The process as claimed in claim 4, wherein the pigment is selected from the group consisting of black iron oxide, ultramarines, red iron oxide, yellow iron oxide, titanium dioxide, zinc oxide cobalt aluminum oxide, chromium oxide green, chromium hydroxide, D&C Black #2, D&C Red 7 Ca Lake, FD&C Yellow 5 Al Lake, FD&C Blue Al Lake, D&C Red 6 Ba Lake, D&C Red 33 Al Lake, FD&C Red 3 Al Lake, D&C Red 27 Al Lake, D&C Red 21 Al Lake, D&C C Red 7 Na Lake, FD&C Yellow 6 Al Lake, D&C Orange Zr Lake, D&C Red 19 Al Lake and D&C Red 21 Al Lake and mixtures thereof.

9. The method as claimed in claim 1, wherein the esterification reaction product is triisostearyl trilinoleate.

10. The composition as claimed in claim 2, wherein the esterification reaction product is triisostearyl trilinoleate.

11. The composition as claimed in claim 3, wherein the esterification reaction product is triisostearyl trilinoleate.

12. The process as claimed in claim 4, wherein the esterification reaction product is triisostearyl trilinoleate.

13. The method as claimed in claim 1, wherein the esterification reaction product is diisostearyl dilinoleate.

14. The composition as claimed in claim 2, wherein the esterification reaction product is diisostearyl dilinoleate.

15. The composition as claimed in claim 3, wherein the esterification reaction product is diisostearyl dilinoleate.

16. The process as claimed in claim 4, wherein the esterification reaction product is diisostearyl dilinoleate.

17. The method as claimed in claim 1, wherein the esterification reaction product is obtained by reacting
(i) a C₁₄-C₂₀ fatty alcohol with
(ii) an unsaturated dimerized or trimerized C₁₆-C₂₀ fatty acid having 2 to 4 unsaturated bonds.

18. The method as claimed in claim 1, wherein the esterification reaction product has the structure: 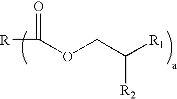
wherein R is the aliphatic hydrocarbon residue of a dimerized or trimerized C₁₆ to C₁₈ fatty acid;
R₁ and R₂ are independently a linear alkyl of 6 to 9 carbons;
a is 2 when R is a dimer acid radical, and
a is 3 when R is a trimer acid radical.

19. The method as claimed in claim 1, wherein the C₁₂-C₂₄ fatty alcohol is a branched chain alcohol that has a C₆-C₁₀ alkyl branch.

20. The method as claim in claim 1, wherein said amount is from oil to 10% by weight based on the total weight of the composition.

21. The method as claimed in claim 1, wherein said amount is from 0.25 to 5% by weight based on the total weight of the composition.

22. The method as claimed in claim 9, wherein said amount is 0.25% by weight based on the total weight of the composition.

23. The method as claimed in claim 13, wherein said amount is 0.25% by weight based on the total weight of the composition.

24. A method for imparting shine to a pigment containing aqueous based cosmetic composition comprising incorporating in such composition an amount of an esterification reaction product of
(i) a C₁₂-C₂₄ fatty alcohol and
(ii) an unsaturated dimerized or trimerized C14-C₂₄ fatty acid, effective to impart shine to the composition as compared to a like composition not containing the esterification reaction product
